(19) United States
(12) Patent Application Publication
(10) Pub. No.: US 2009/0259141 A1
(43) Pub. Date: Oct. 15, 2009

(54) STEERABLE TOOL GUIDE FOR USE WITH FLEXIBLE ENDOSCOPIC MEDICAL DEVICES

(75) Inventors: Richard C. EWERS, Fullerton, CA (US); Arvin T. CHANG, West Covina, CA (US); Robert A. VAUGHAN, Leander, TX (US)

Correspondence Address: LEVINE BAGADE HAN LLP, 2400 GENG ROAD, SUITE 120, PALO ALTO, CA, 94303, US

(73) Assignee: USGI Medical, Inc., San Clemente, CA (US)

(21) Appl. No.: 12/407,993

(22) Filed: Mar. 20, 2009

Related U.S. Application Data
(60) Provisional application No. 61/038,642, filed on Mar. 21, 2008.

(51) Int. Cl. A61B 1/018; A61B 10/04

(52) U.S. Cl. 600/562; 600/106

(57) ABSTRACT

An articulatable, steerable tool guide includes a maneuverable head subassembly, a flexible or rigid insertion tube subassembly, and a handle subassembly. The tool guide defines at least one inner lumen extending through the length of the tool guide, with each such lumen being adapted to receive a flexible endoscopic medical device.

### 1. CROSS-REFERENCE TO RELATED APPLICATIONS

This application claims benefit of priority to U.S. Provisional Patent Application No. 61/038,642, filed on Mar. 21, 2008, the content of which is incorporated herein by reference in its entirety.

### 2. BACKGROUND

The invention relates to flexible endoscopic surgical devices and, more particularly, to an articulatable tool guide that accommodates and articulates various flexible endoscopic surgical tools and other devices, or that provides steering and articulation for integrated end effectors having the functional capabilities of endoscopic tools and devices.

Flexible endoscopic medical devices (FEMD) have been and continue to be developed to assist in minimally invasive endoscopic surgery. One limitation of most FEMDs is that their distal ends (surgical end) cannot be independently steered. The devices are limited in their positional degrees of freedom to the axis of the endoscope's lumen, with the result that the user must rely on the endoscope to steer and maneuver the device. These limitations also restrict the user to viewing the motion of the FEMD to the same line of sight as the endoscope. The desire to perform more challenging minimally invasive surgical procedures has increased the demand for FEMDs that are independently maneuverable.

### 3. SUMMARY

An articulatable, steerable tool guide is disclosed. The tool guide includes a maneuverable distal head assembly, a flexible or rigid insertion tube assembly, and a handle assembly. The tool guide defines at least one inner lumen extending through the length of the tool guide. During an endoscopic procedure, the tool guide is inserted into the lumen of an endoscope or an endoscopic device, which is advanced endoscopically to a target location within the body of a patient undergoing an endoscopic diagnostic and/or therapeutic procedure. In alternative embodiments, the tool guide is used independently, without being inserted into an endoscope or endoscopic device. Another FEMD can then be advanced, manipulated, and withdrawn through the inner lumen of the tool guide. Advantageously, multiple FEMDs can be sequentially inserted, manipulated, and withdrawn while the tool guide is left in place in order to perform procedures requiring functionality from more than one FEMD. As a result, the device functions as a steerable guide that enables other FEMDs to be maneuvered independently of an endoscope.

In several embodiments, the steering capability of the tool guide comprises several useful motions. For example, in an embodiment, the steering motion is a single curve. The curve is controllable in a single plane, or in multiple planes. In some embodiments, a single plane curve is rotated to align with alternate planes by applying a torque force to the tool guide.

In other embodiments, the steerable tool guide is capable of being articulated in more than a single curve. For example, in some embodiments, the tool guide is articulated to take the form of a compound curve. In this manner, an FEMD that is contained within the inner lumen of the tool guide is routed on a path away from the longitudinal axis of the endoscope and then back into the viewing field at a selected angle with respect to the longitudinal axis of the endoscope. Thus, the tool guide is capable of defining a path for an FEMD that ranges from being substantially aligned with the longitudinal axis of the endoscope to being an “S”-shape or a “Crooked” shape. In several embodiments, the FEMD is routed into a position at a forward pointing angle directed at the longitudinal axis of the scope but located at a position that does not cross the longitudinal axis. In this manner, two tool guides are positioned so that they are able to work in conjunction on an item of interest that is located central to the field of vision.

In several embodiments, the steerable tool guide provides planar stability. The tool guide is capable of forming the compound curve described above and also to have planar stability perpendicular to the “shaping” plane. This is useful in that a shaped tool guide is able to be rotated with respect to the longitudinal axis defined by its shaft to generate “flipping” or lifting actions. Similarly, in several embodiments, the tool guide has the ability to lock out in the shaped form. This feature provides stability in linear translation so that an articulated tool guide is able to push or pull by translation of the shaft.

In several additional embodiments, the tool guide is able to be utilized with FEMDs having sizes, shapes, and other physical attributes and properties that are common to many current FEM Ds. By way of non-limiting example, in several embodiments, the tool guide has an OD in the range of from about 3 mm to about 5 mm, and an inner lumen having an ID of from about 1.5 mm to about 3.5 mm. At these dimensions, the inventors have found many commercially available FEMDs that are labeled “2.8 mm” that will fit, for example, in a 2.4 mm ID measured lumen. Several examples of FEMDs suitable for use in association with the tool guide include, but are not limited to: biopsy cups, graspers, scissors, snares, needles, multi prong graspers, electrocautery instruments, retrieval baskets, and catheters. FEMDs may be standalone instruments or instruments made custom to work in conjunction with the tool guide. In the tool guide embodiments that are steerable, it is important for the FEMD to have a flexible or semi-flexible shaft in the region that is intended to be formed into the steered curved path.

In several embodiments, the handle assembly is configured to both control the motion of the distal head assembly and to accommodate a variety of FEMDs. Once an FEMD is inserted into the inner lumen of the tool guide, the FEMD can be held in a fixed position relative to the tool guide. By activating a turn knob, the distal end of the FEMD can be made to articulate. By translating a telescoping tube on the handle, the FEMD can be made to translate with respect to the tool guide.

The handle provides the capability of proximal control of the actuation of the articulating distal end. This is accomplished in some embodiments with a binary control to take the distal end from straight to shaped or, in other embodiments, with a continuously positioning ratchet-type actuation. In an embodiment, the distal shaping end is controlled with a rotating knob and a threaded shaft. Rotation of the knob drives the shaft. The lead of the thread is such that the knob cannot be driven in reverse by the resistive force of the distal end.

In several embodiments, the actuator has a telescoping feature. Many currently available FEMDs are flexible along the entire shaft. To introduce these FEMDs down a channel, the user must hold the shaft in close proximity to the entrance of the channel. Advancement is only accomplished by multiple, short, serial advancements. In several embodiments of the present tool guide, the actuator has a telescoping sleeve. The FEMD can be positioned in the tool guide and fixed to the sleeve. The sleeve is stable and may translate relative to the actuator so that it can be advanced and withdrawn. In this fashion, the FEMD can be advanced and withdrawn without the need for the multiple short, serial advancements described above. The sleeve can also be constructed so that the fixation point is able to rotate. In this manner, instruments can be aligned in rotation while still maintaining a fixed translational position with respect to the telescoping sleeve. In addition, once the tool guide head assembly is articulated and/or steered to a desired orientation, the FEMD is able to be advanced and withdrawn in order to reach objects that are located beyond the tool guide but within the articulated path and extended reach of the FEMD.

It is also advantageous in some embodiments that the handle provide electrical insulation. Electrical current could be generated directly by electro-surgical tool end-effectors accidentally coming into contact with (or come within close proximity of) the conductive components of the tool guide, thus creating a short. Capacitive coupling between the electrical FEMD and the insertion shaft assembly of the tool guide may also be another source of current leakage. One way to minimize this type of potentially harmful current leakage is to insulate the handle from the conductive components of the insertion shaft subassembly and distal head subassembly.

In several alternative embodiments, a variety of miniature surgical tool tips or end-effectors are attachable to the distal tip of the tool guide. The tool guide may then function as an articulatable multifunction FEMD with interchangeable surgical tool tips. In other embodiments, the tool tips are configured to be permanently coupled to the tool guide.

### 4. BRIEF DESCRIPTION OF THE DRAWINGS

FIG. 1 is a side view of a tool guide assembly.

FIGS. 2 and 3 are a side view and a perspective view, respectively, of a flexible endoscopic medical device coupled with the tool guide assembly shown in FIG. 1.

FIG. 4A is a side view of an embodiment of a head subassembly of the tool guide assembly of FIG. 1 shown in a straight on-axis configuration.

FIG. 4B is a side view of the head subassembly of FIG. 4A shown in an articulated configuration.

FIGS. 4C-4E are side views of a manifold bushing, a swivel, and a center bushing, respectively, of the head subassembly shown in FIG. 4A.

FIGS. 5A and 5B are side views of another embodiment of a head subassembly shown in a straight on-axis configuration and an articulated configuration, respectively.

FIGS. 6A and 6B are side views of still another embodiment of a head subassembly shown in a straight on-axis configuration and an articulated configuration, respectively.

FIGS. 7A and 7B are side views of additional embodiments of a head subassembly shown in an articulated configuration.

FIG. 8 is a cross-sectional view of an embodiment of an insertion tube assembly of the tool guide assembly shown in FIG. 1.

FIGS. 9 and 10 are a length element view and an isometric view, respectively, of an embodiment of a main body tube of the insertion tube assembly shown in FIG. 8.

FIG. 11 is a schematic view of a handle assembly of the tool guide assembly of FIG. 1.

FIG. 12 is a perspective view of a flexible endoscopic medical device coupled with the handle assembly shown in FIG. 11.

FIGS. 13A-C are perspective illustrations showing tool guide assemblies and flexible endoscopic medical devices deployed through an endoscopic access device.

### 5. DETAILED DESCRIPTION

During use of conventional FEMDs for diagnosing or treating human patients, the FEMD is advanced into the human body via the tool lumen of an endoscope or an endoscopic device. In such a configuration, the FEMD must rely on the maneuverability of an endoscope or endoscopic device for any type of tool tip positioning during a diagnostic or therapeutic procedure. This restriction greatly limits the capability of the surgeon performing a complex minimally invasive procedure. Furthermore, the surgical field of view (FOV) as seen through an endoscope must be maintained as unobstructed as possible during minimally invasive surgical procedures. Where possible, movement of any FEMD is preferably achieved in a manner that does not obstruct or limit the FOV. Accordingly, providing a stable platform through which an FEMD may be maneuvered independently of an endoscope or other endoscopic access device will enhance the capabilities of the surgeon.

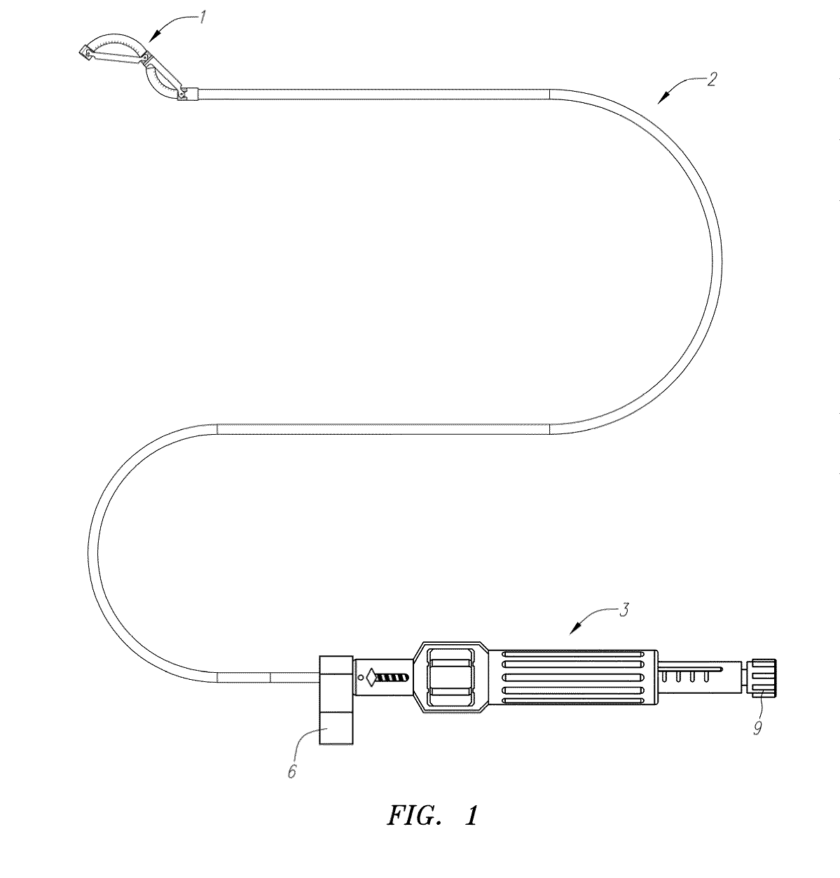

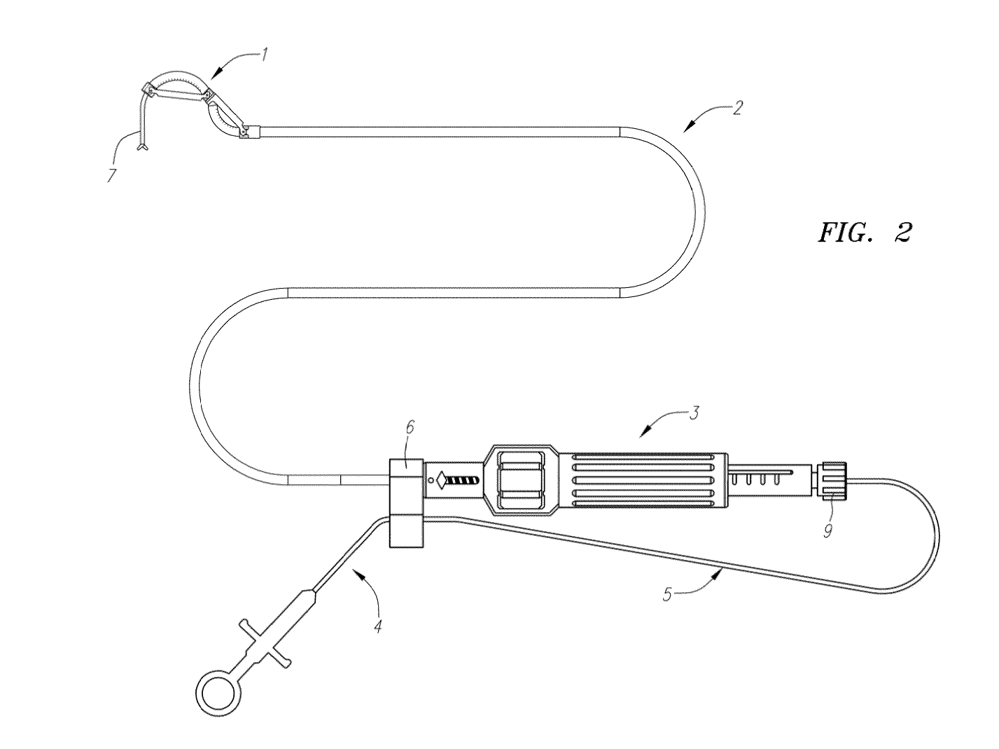

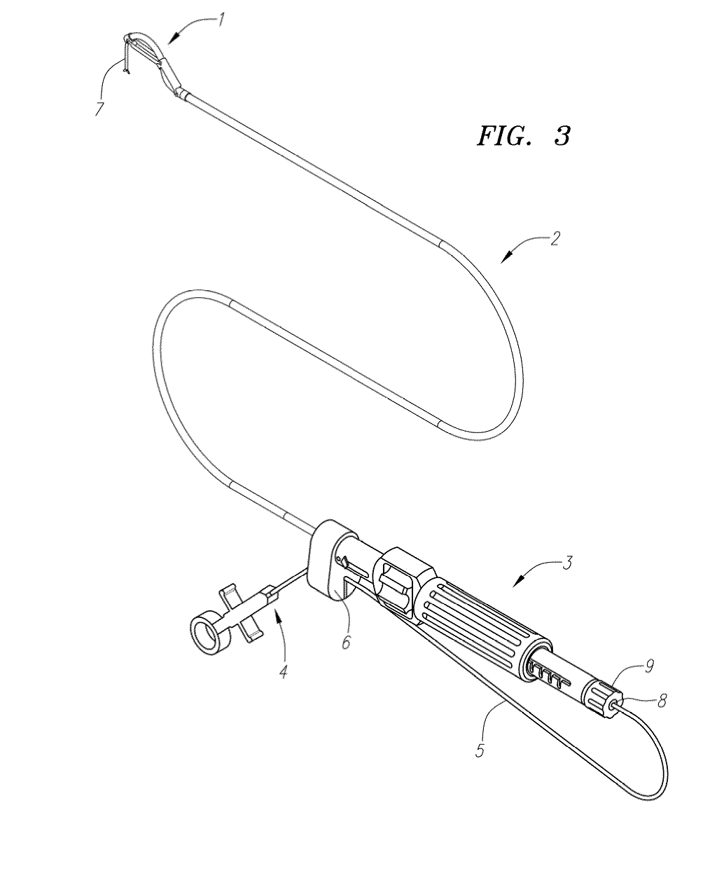

A tool guide assembly capable of providing this capability for FEMDs is illustrated in FIG. 1. The tool guide includes a handle subassembly 3, an insertion tube subassembly 2, and a steerable head subassembly 1. FIG. 2 illustrates an embodiment of the tool guide in which a FEMD 4 is coupled to the tool guide. A shaft 5 of the FEMD 4 is inserted through the handle inlet port 8. The flexible or rigid shaft 5 is secured in place using a securing mechanism, such as a Tuohy Borst adapter 9 or other actuatable iris valve or similar mechanism providing a substantially fixed relationship between the tool guide and the FEMD. An optional tool holder 6 made of a conformable material may be utilized to manage the excess length of the FEMD 4.

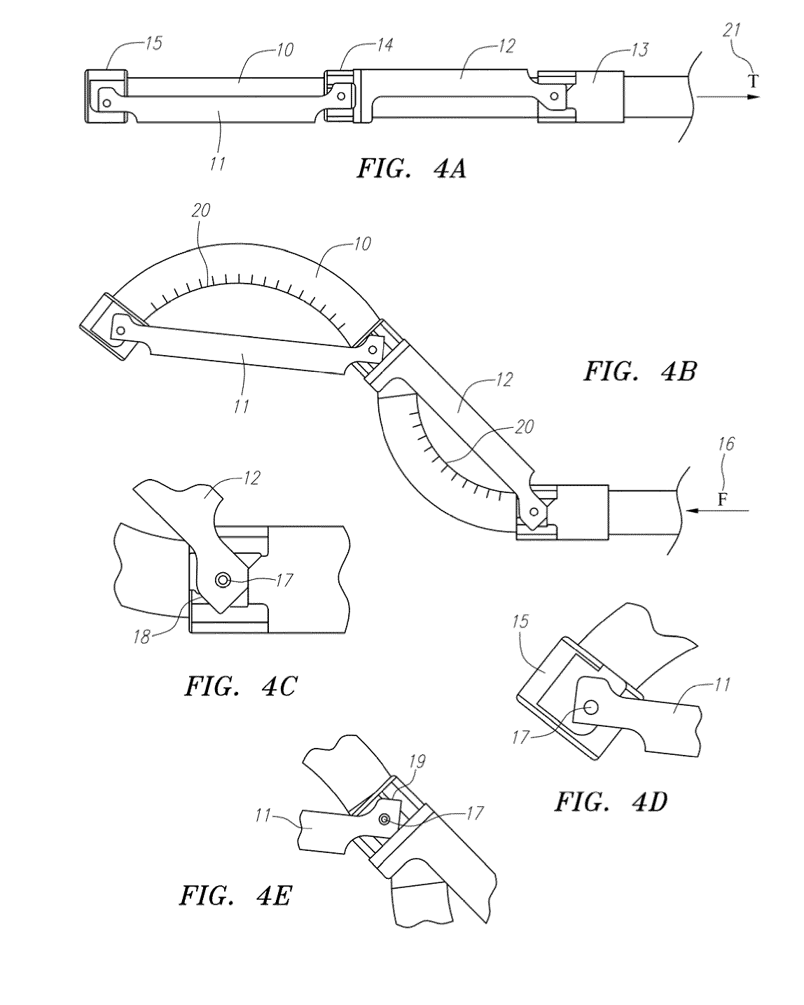

The head subassembly 1 includes an S-shape formable head tube 10, distal and proximal linkage arms 11 and 12, a manifold bushing 13, a center bushing 14, and a swivel 15. FIG. 4A shows the head assembly 1 in a straight on-axis configuration. Activation of the head subassembly 1 into an articulated configuration is achieved by applying a compression force 16 on the S-shaped head tube 10. FIG. 4B shows the head assembly 1 in an articulated configuration. The S-shaped head tube 10 has a series of slits 20 that are spaced and configured in a manner to achieve the bend geometry that is desired. When a compression force 16 is applied, the S-shaped head tube 10 buckles against the linkages 11, 12, 13 to a predetermined “S” shape.

Referring to FIG. 4C, the linkage arms 11 and 12 are able to freely rotate about a pin 17 located on each of the bushings 13, 14, and 15. Upon application of a compression force 16, the distal linkage 11 will rotate counter clockwise with respect to the center bushing 14 and the proximal linkage 12 will rotate clockwise with respect to the manifold bushing 13 synchronously. Rotation of the linkages 11 and 12 will terminate despite an increase in compression force 16 once the linkages 11 and 12 come in contact with respective mechanical stops 19 and 18. This interaction locks Out the head assembly 1 into a rigid articulated configuration.

Conversely, applying a tensile force 21 will cause the head subassembly 1 to return to its straight configuration. Once in the articulated configuration, applying a tensile force 21 will initially cause the proximal linkage 12 to rotate counter-clockwise until it is in the straight configuration, followed sequentially by the clockwise rotation of the distal linkage 11. Thus, by controlling compression and tensile forces 16 and 21, the user is able to control the positioning of the distal head subassembly 1. This enables the user to steer and maneuver the tool tip 7 of an FEMD 4.

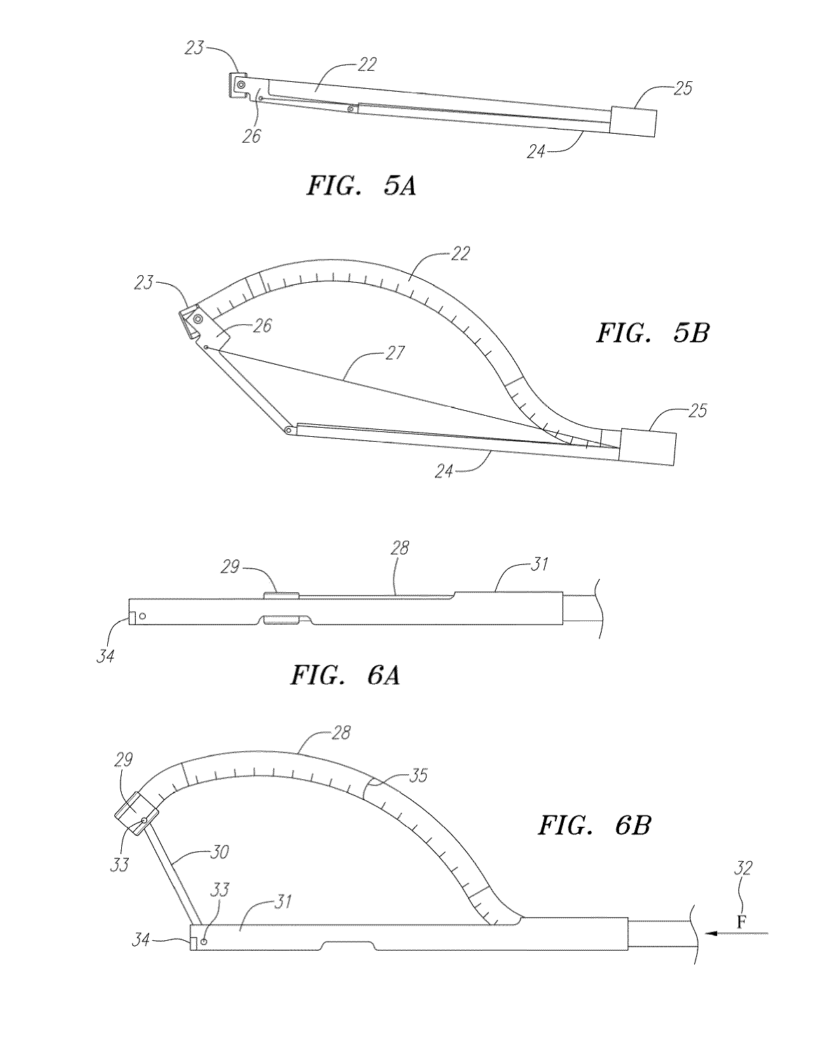

FIGS. 5A and 5B illustrate another embodiment of the head subassembly 1. In this embodiment, a laser cut tube 22 is fixed in place with respect to a base bushing 25 and a swivel bushing 23. A linkage arm 26 is free to rotate about its pivot point where it is pivotably attached (e.g., by a pin or similar mechanism) to a strut 24. The strut 24 is fixed with respect to the base bushing 25. A pull wire 27 is fixed with respect to the linkage arm 26, but free to translate through the bushing 25. Articulation and steering of the head subassembly 1 is achieved by applying tension on the pull wire 27. Tension on the pull wire 27 causes the linkage arm 26 to move clockwise and causes the swivel 23 to pivot. The laser cut tube 22 includes slots that have sizes and shapes such that the laser cut tube 22 will take a certain desired shape upon compression.

FIGS. 6A and 6B show still another embodiment of the head subassembly 1. In this embodiment, a laser cut tube 28 is fixed at its distal end to a swivel 29 but is free to translate through a strut 31. A linkage 30 is free to rotate and connected via pins 33 to the swivel 29 and the strut 31. Articulation of the head sub assembly is achieved by applying an axial compression force 32 to the laser cut tube 28. Upon application of the force 32, the laser Cut tube 28 bends into a certain curvature as defined by the shapes, sizes, and patterns defined by the slits 35 formed in the tube. Bending and advancement of the laser cut tube 28 also causes the linkage arm 30 to rotate counterclockwise until it comes into contact with a mechanical stop 34. The swivel 29 also rotates counterclockwise accordingly.

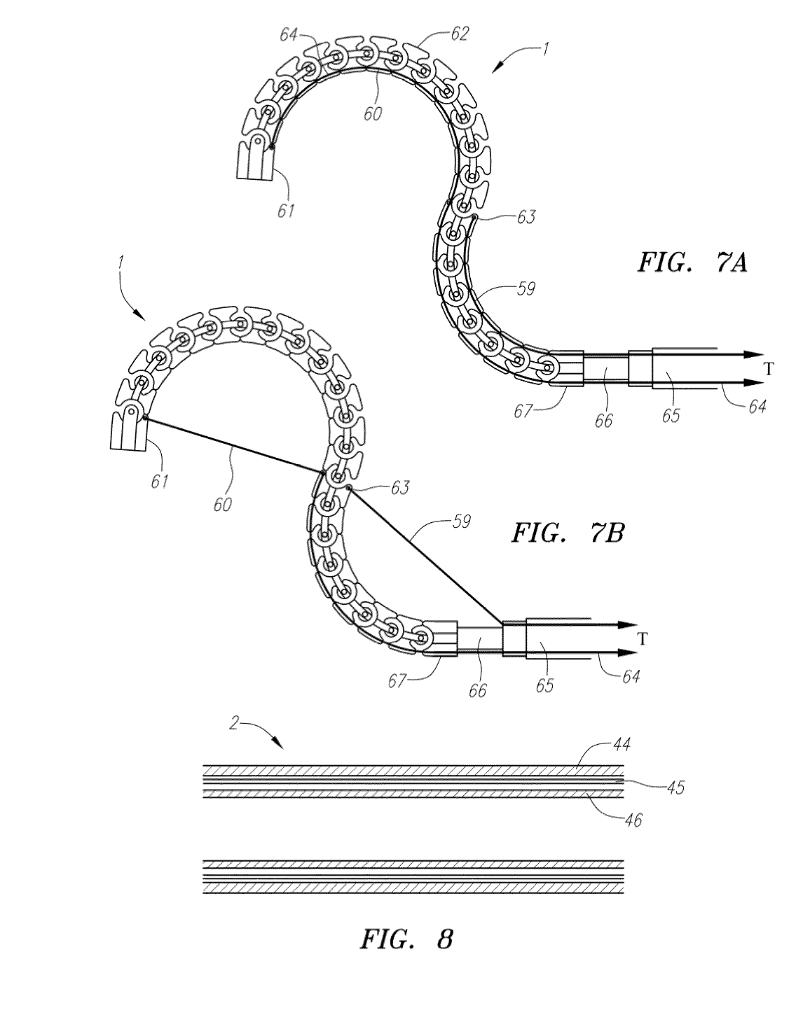

FIGS. 7A and 7B show additional embodiments of the head subassembly 1. In these embodiments, a series of pinned links 62 define the distal end of the tool guide. Each pair of adjacent links is pinned together at a pin point 64, allowing each link 62 to rotate with respect to its adjacent links 62. In the FIG. 7A embodiment, a first pull wire 60 runs through a throughhole provided in each pinned link 62. One end of the first pull wire 60 is affixed to the distal link 61. A second pull wire 59 also runs along the throughhole in several of the proximally located pinned links 62, except that it terminates and is affixed to a transition link 63 located proximally of the distal link 61. Applying tension 64 on the first pull wire 60 causes the full length of the linked head subassembly to articulate in a counter-clockwise direction. Applying tension 64 on the second pull wire 59 causes the proximal portion of the head subassembly to articulate in a clockwise direction. Applying tension 64 simultaneously to both pull wires 60 and 59 will result in simultaneous counter-clockwise articulation of the full length of the subassembly and clockwise articulation of the proximal portion of the subassembly, as illustrated in FIG. 7A. Alternatively, the pull wires 60 and 59 can be configured such that they are partially exposed and not fully enclosed by each pinned link 62. FIG. 7B illustrates an embodiment in which the pull wires 60 and 59 are partially exposed. Positioning the pull wires 59 and 60 in this configuration provides additional mechanical advantage (leverage) and provides for a more rigid head subassembly 1. Furthermore, in still other embodiments, the pull wires 59 and 60 are not directly pulled to actuate the head subassembly 1. In these other embodiments, the pull wires 59 and 60 are affixed to a hub 65. The push rod 66 is attached to a base link 67 but is free to slide within the hub 65. By pushing the push rod 66 forward, a tension 64 is indirectly created to thereby simultaneously actuate both pull wires 59 and 60.

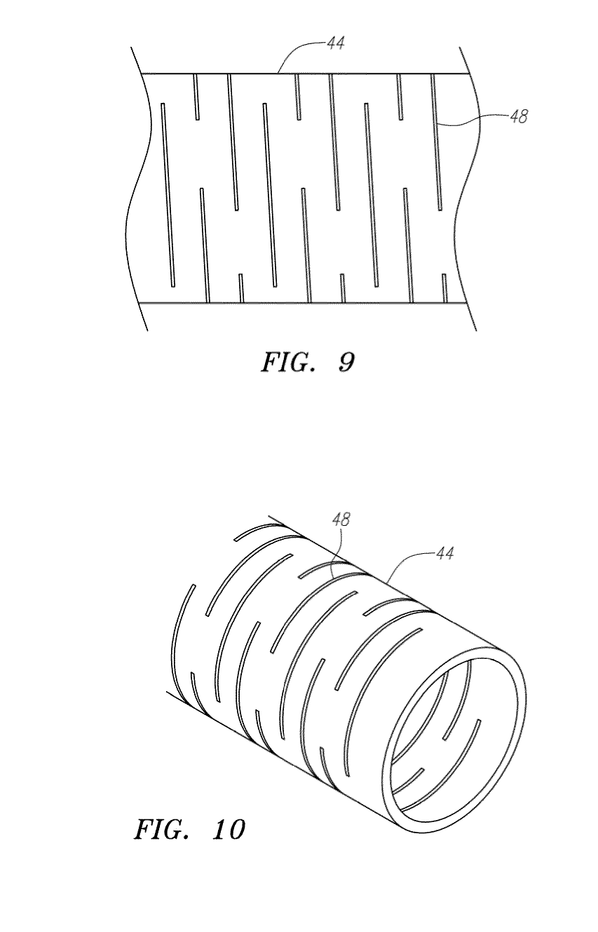

FIG. 8 is a cross section view of an embodiment of the insertion tube assembly 2. The insertion tube assembly 2 includes a main body tube 44, a liner 45, and a force transmission tube 46. The main body tube 44 may be flexible or rigid, or it may have regions of varying flexibility and rigidity. The main body tube 44 may comprise a braided polymer tube or any other torqueable tube subassembly. FIGS. 9 and 10 are a length element view and isometric view disclosing an embodiment of a main body tube 44. The tube 44 is formed of a resilient material such as stainless steel (though not limited to stainless steel) tubing having a pattern of slits 48 formed therein. In the embodiment shown in FIGS. 9 and 10, the slit pattern 48 includes a spiral pattern with a desired pitch and cut angle. Different patterns of slits 48 will have the result of providing different mechanical properties for the main tube 44. In some embodiments, a liner 45 comprising a separate tube with lubricious properties or a polymer layer or coating is coupled to the ID of the main body tube 44 or the OD of the force transmission tube 46. The force transmission tube 46 is able to slide freely within the lumen defined by the ID of the main body tube 44. The inner lumen of the force transmission tube 46 serves as a conduit for any FEMD 4.

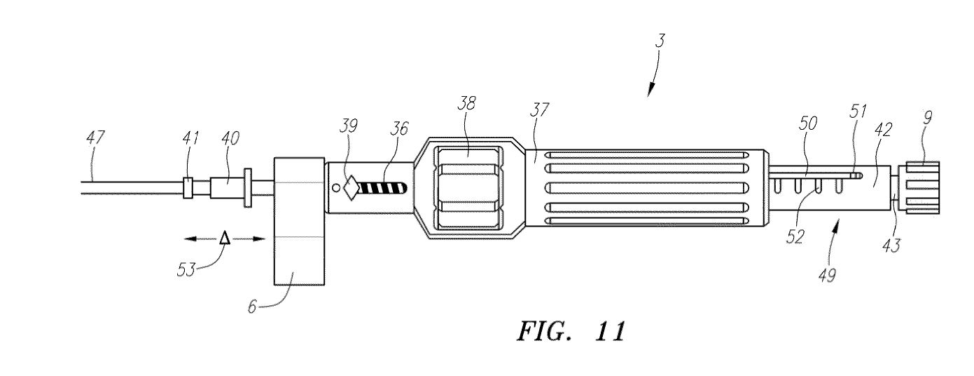

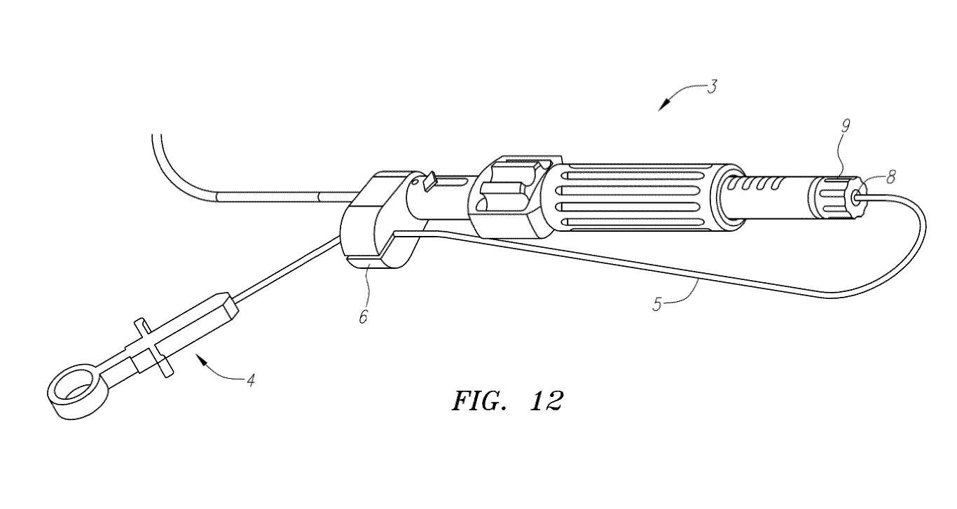

FIG. 11 is a schematic view of the handle assembly 3. A lead screw 36 is enclosed in the main handle body 37. The lead screw 36 is able to translate about the axis of the main handle body 37. Translational actuation of the lead screw 36 is accomplished by rotation of the turn knob 38. In an embodiment, the lead screw 36 is coupled to a proximal end of the force transmission tube 46, and distal end of the force transmission tube 46 is coupled to the laser cut tube 10. In this embodiment, actuation of the lead screw 36 produces a compressive force 16 that is transmitted via the transmission tube 46 to the laser cut tube 10, thus causing the head subassembly 1 to be articulated. Actuation of the lead screw 36 in the opposite direction straightens the head subassembly 1 to its un-articulated state. Furthermore, an optional indicator 39 may be attached to the surface of the lead screw 36. The indicator 39 moves with the lead screw 36 to provide a visual indication of the degree of articulation of the head subassembly 1 as a function of the position of lead screw 36.

In the embodiment shown in FIG. 11, a telescoping subassembly 49 is included in the handle assembly 3. The telescoping subassembly 49 includes an inlet tube 43, a telescoping tube 42, and a touhy borst adapter 9. During use, a user inserts the distal end of an FEMD 4 into and through the inlet port 8. Once the FEMD 4 is inserted into place, the touhy borst adapter 9 is used to lock the FEMD 4 in place relative to the tool guide. The touhy borst adapter 9 is attached to the telescoping tube 42, but is free to rotate about the telescoping tube 42. The telescoping tube 42 is free to translate about the inlet tube 43. Translation of the telescoping tube 42 is limited to the length of the sliding track 50. A pin 51 is fixed in place on the inlet tube 43 and resides in slots provided in the sliding track 50. In this embodiment, a user can telescope the telescoping tube 42 to translate an FEMD 4 that is fixed in place by the touhy borst adapter 9 about the inner lumen of the tool guide. Further, the user may decide to turn the telescoping tube 42 in a manner such that the pin 51 will lock in place to a side track 52. This interaction locks the telescoping tube 42 and prevents the telescoping tube 42 from translating about the inlet tube 43. This in turn prevents an FEMD 4 from moving with respect to the tool guide. A plurality of side tracks 52 enable multiple locking positions. The telescoping action provides the ability for the FEMD to extend into or out of the steerable tip of the tool guide, thereby providing additional position functionality for the working (distal) end of the FEMD.

In some embodiments, an optional leashing collar 40 is employed. The leashing collar 40 is able to slide freely about a rigid proximal portion 47 of the shaft. A stop collar 41 is affixed to the rigid shaft 47. During use, the leashing collar 40 is locked in place relative to the inlet port of an endoscope or endoscopic device. Once the leashing collar 40 is locked in place, translation of the tool guide through the lumen of the endoscope is limited to delta 53, as defined by the position of the stop collar 41 and the tool holder 6.

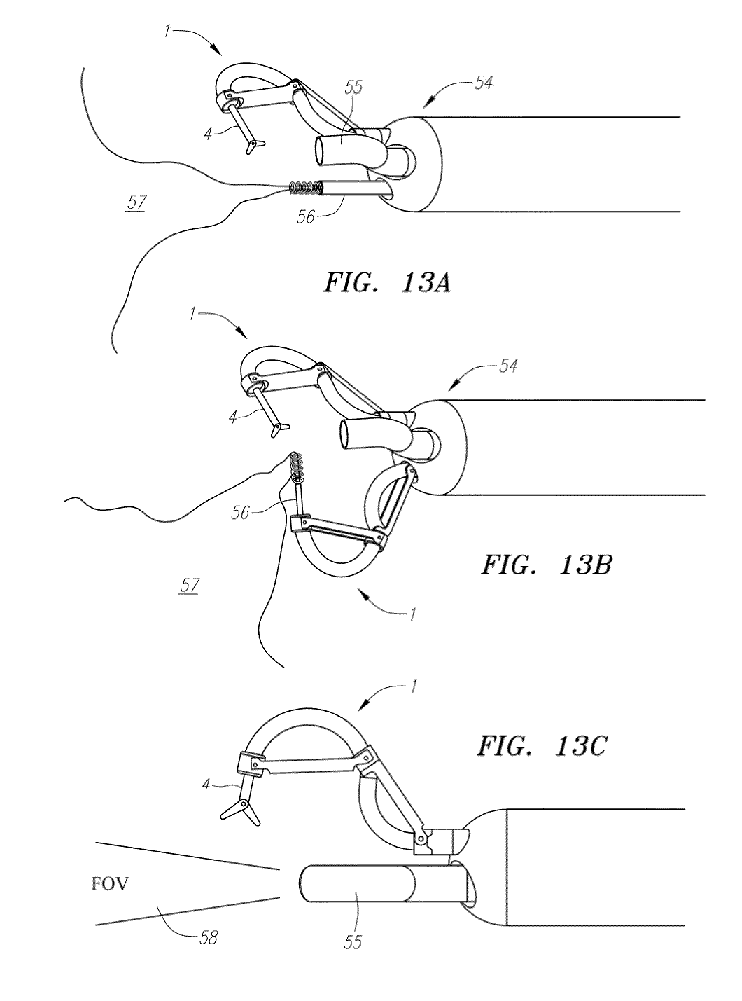

In several embodiments, the tool guide is deployed through an endoscopic tool deployment system, such as the TransPort™ multi-lumen endoscopic access device developed by USGI Medical, Inc. of San Clemente, Calif. Examples of endoscopic access devices and systems are described in further detail in U.S. patent application Ser. Nos. 10/797,485, filed Mar. 9, 2004; 11/750,986, filed May 18, 2007; and 12/061,951, filed Apr. 2, 2008, each of which is incorporated herein by reference in its entirety. FIG. 13A is a schematic view of an articulated head subassembly 1 that is exposed outside the distal end of an endoscopic access device 54. In this embodiment, forcing the tool guide back through the tip of the access device will cause damage to the tool guide or the access device. Utilizing the leashing collar 40 will prevent damage from occurring. Locking the leashing collar 40 in place relative to the inlet of the access device prevents the head subassembly 1 from retracting into the tip of the device 54 when the tool guide is being advanced and retracted.

FIG. 13A through 13C show several embodiments of tool guides in use. In FIG. 13A, one tool guide is used in conjunction with a helical grasping tool 56 and an endoscope 55. The helical grasping tool 56 is used to engage tissue 57 while the tool guide is used to steer a cutting tool type FEMD 4. Alternatively, in the embodiment shown in FIG. 13B, two tool guides are used to steer two endoscopic tools, including a helical grasper 56 and a cutting tool 4. FIG. 13C illustrates the compound articulation capability of the head subassembly 1. By articulating outside the longitudinal axis of the endoscope 55, the field of view 58 of endoscope 55 is not obstructed.

Although various illustrative embodiments are described above, it will be evident to one skilled in the art that various changes and modifications are within the scope of the invention. It is intended in the appended claims to cover all such changes and modifications that fall within the true spirit and scope of the invention.

What is claimed is:

1. A tool guide for a flexible endoscopic device, comprising:
a handle;
a tubular sheath having a proximal end and a distal end, with the proximal end being attached to the handle;
a force transmission tube extending substantially coaxially and slidably within the tubular sheath and having a proximal portion located within the tubular sheath and a distal portion extending beyond the distal end of the tubular sheath, with the distal portion of the force transmission tube including a plurality of circumferential slots, and with the distal portion having an on-axis configuration in which the distal portion is substantially longitudinally aligned with the proximal portion and an articulated configuration in which the distal portion is not substantially longitudinally aligned with the proximal portion;
a first bushing disposed on the distal portion of the force transmission tube;
a first substantially rigid linkage arm having a first end pivotably connected to the distal end of the tubular sheath and a second end pivotably connected to the first bushing; and
a second substantially rigid linkage arm having a first end pivotably connected to the first bushing and a second end pivotably connected to a distal end of the force transmission tube.

2. The tool guide of claim 1, wherein the distal portion of the force transmission tube is in the form of a compound curve when in the articulated configuration.

3. The tool guide of claim 2, wherein the distal portion of the force transmission tube is substantially in the form of an “S” shape when in the articulated configuration.

4. The tool guide of claim 1, further comprising a swivel disposed at or near the distal end of the distal portion of the force transmission tube, and wherein the second end of the second substantially rigid linkage arm is pivotably connected to the swivel.

5. The tool guide of claim 1, further comprising a stop member located on said first bushing and having a stop surface that engages a portion of the second substantially rigid linkage arm when the second substantially rigid linkage arm pivots around the first bushing.

6. The tool guide of claim 1, further comprising a flexible endoscopic medical device extending substantially coaxially and slidably within the force transmission tube and having an end effector extending beyond the distal end of the force transmission tube.

7. The tool guide of claim 6, wherein said flexible endoscopic medical device comprises a grasper.

8. The tool guide of claim 6, wherein said flexible endoscopic medical device comprises an electrocautery instrument.

9. The tool guide of claim 6, wherein said flexible endoscopic medical device comprises a scissors.

10. The tool guide of claim 6, wherein said flexible endoscopic medical device comprises a biopsy cups.

11. The tool guide of claim 1, wherein the distal portion of the force transmission tube is transitioned from the on-axis configuration to the articulated configuration by application of a distally-directed compression force on the force transmission tube.

12. The tool guide of claim 1 further comprising an actuator located on the handle and operatively coupled with the force transmission tube, the actuator having a first configuration corresponding with the on-axis configuration of the distal portion of the force transmission tube and a second configuration corresponding with the articulated configuration of the distal portion of the force transmission tube.

13. The tool guide of claim 12, wherein said actuator comprises a lead screw.

14. The tool guide of claim 12, further comprising a telescoping tube that is slidably associated with an inlet tube of the handle and that is attached to a flexible endoscopic medical device extending substantially coaxially and slidably within the force transmission tube, the flexible endoscopic medical device having an end effector extending beyond the distal end of the force transmission tube.

15. The tool guide of claim 14, further comprising an iris valve located on the telescoping tube, with the flexible endoscopic medical device extending through a seal defined by the iris valve.

16. An endoscopic tool deployment system comprising:
an endoscopic access device including an elongated shaft having at least one lumen extending through at least a portion of the shaft; and
a tool guide extending through the at least one lumen of the endoscopic access device, the tool guide comprising:
a handle;
a tubular sheath having a proximal end and a distal end, with the proximal end being attached to the handle;
a force transmission tube extending substantially coaxially and slidably within the tubular sheath and having a proximal portion located within the tubular sheath and a distal portion extending beyond the distal end of the tubular sheath, with the distal portion of the force transmission tube including a plurality of circumferential slots, and with the distal portion having an on-axis configuration in which the distal portion is substantially longitudinally aligned with the proximal portion and an articulated configuration in which the distal portion is not substantially longitudinally aligned with the proximal portion;
a first bushing disposed on the distal portion of the force transmission tube;
a first substantially rigid linkage arm having a first end pivotably connected to the distal end of the tubular sheath and a second end pivotably connected to the first bushing; and
a second substantially rigid linkage arm having a first end pivotably connected to the first bushing and a second end pivotably connected to a distal end of the force transmission tube.

17. The endoscopic tool deployment system of claim 16, further comprising a flexible endoscopic medical device extending substantially coaxially and slidably within the force transmission tube and having an end effector extending beyond the distal end of the force transmission tube.

18. The endoscopic tool deployment system of claim 16, further comprising an actuator located on the handle of the tool guide and operatively coupled with the force transmission tube, the actuator having a first configuration corresponding with the on-axis configuration of the distal portion of the force transmission tube and a second configuration corresponding with the articulated configuration of the distal portion of the force transmission tube.

19. A method for articulating a flexible endoscopic medical device, comprising:
providing a tool guide comprising a tubular sheath having a proximal end and a distal end and a force transmission tube extending substantially coaxially and slidably within the tubular sheath, the force transmission tube having a proximal portion located within the tubular sheath and a distal portion extending beyond the distal end of the tubular sheath, with the distal portion of the force transmission tube including a plurality of circumferential slots, and with the distal portion having an on-axis configuration in which the distal portion is substantially longitudinally aligned with the proximal portion and an articulated configuration in which the distal portion is not substantially longitudinally aligned with the proximal portion;
applying a distally-directed compression force on the force transmission tube while restraining distal movement of the distal end of the force transmission tube, thereby transitioning the distal portion from the on-axis configuration to the articulated configuration; and
translating the flexible endoscopic medical device through a lumen defined by the tool guide such that an end effector of the flexible endoscopic medical device extends beyond the distal end of the force transmission tube.

20. The method of claim 19, wherein the distal portion of the force transmission tube is in the form of a compound curve when in the articulated configuration.

21. The method of claim 20, wherein the distal portion of the force transmission tube is substantially in the form of an “S” shape when in the articulated configuration.

22. The method of claim 19, further comprising endoscopically advancing the tool guide to a target location within the body of a patient.
